(19) United States
(12) Patent Application Publication
(10) Pub. No.: US 2024/0167003 A1
(43) Pub. Date: May 23, 2024

(54) MEMBRANE FILTRATION APPARATUS, CONCENTRATION APPARATUS, AND CONCENTRATION METHOD

(71) Applicant: Yokogawa Electric Corporation, Tokyo (JP)

(72) Inventors: Yasuhiro MATSUI, Musashino-shi, Tokyo (JP); Hiroyuki KATAYAMA, Bunkyo-ku, Tokyo (JP)

(21) Appl. No.: 18/283,937

(22) PCT Filed: Jan. 31, 2022
(86) PCT No.: PCT/JP2022/003673

§ 371 Date: Sep. 25, 2023

(30) Foreign Application Priority Data
Mar. 26, 2021 (JP) 2021-054155

(51) Int. Cl. C12N 7/02; B01D 65/06; B01D 69/02

(52) U.S. Cl. CPC C12N 7/02; B01D 65/06; B01D 69/02; B01D 2325/0283; B01D 2325/26

(57) ABSTRACT

A membrane filtration apparatus 110 including a membrane 113 that is made from an acid-resistant material, has pores having a pore size capable of capturing microorganisms, and has a surface that has a negative charge in a neutral to alkaline range.

### CROSS-REFERENCE TO RELATED APPLICATION

The present application claims priority to and the benefit of Japanese Patent Application No. 2021-054155 filed Mar. 26, 2021, the entire contents of which are incorporated herein by reference.

### TECHNICAL FIELD

The present disclosure relates to a membrane filtration apparatus, a concentration apparatus, and a concentration method.

### BACKGROUND

Negatively charged membrane methods are known as conventional treatment methods for detecting viruses in water. For example, Patent Literature (PTL) 1 describes a method including producing a purified virus solution from a sample solution, concentrating the purified virus solution by a negatively charged membrane method or the like, and detecting a virus by extracting nucleic acid. The sample solution may be from environmental water such as river water, lake water, seawater, rainwater, and the like, drinking water such as well water, tap water, bottled water, and the like, sewage water, wastewater, pool water, water used in industry such as agricultural water, industrial water, refrigerant water, and the like.

### CITATION LIST

### Patent Literature

PTL 1: JP 2015-195756 A

### SUMMARY

### Technical Problem

In a negatively charged membrane method, the negatively charged membrane needs to be replaced each time filtration is performed to secure sample water filtration performance. However, the replacement of the negatively charged membrane needs to be done manually, which requires time and labor. Further, the cost of purchasing negatively charged membranes for replacement and the time taken from purchase to receipt are required.

It would be helpful to provide a membrane filtration apparatus, concentration apparatus, and a concentration method that improve the convenience of processing by the negatively charged membrane method.

### Solution to Problem

According to at least one embodiment, a membrane filtration apparatus comprises a membrane that is made from an acid-resistant material, has pores having a pore size capable of capturing microorganisms, and has a surface that has a negative charge in a neutral to alkaline range. Accordingly, after treatment by the negatively charged membrane method using the membrane filtration apparatus, the membrane filtration apparatus becomes usable again by washing with a strong acid. Therefore, convenience is increased by reducing the time and labor required to replace a filtration membrane, as well as reducing the cost of purchasing negatively charged membranes for replacement and the time taken from purchase to receipt.

According to an embodiment, the membrane is made from an acid-resistant material that withstands pH 2 or lower. Accordingly, the membrane may be washed with a solution of strong acid to dissolve substances of microbial origin.

According to an embodiment, the membrane filtration apparatus further comprises a pressure vessel that is pressure resistant, wherein the membrane is held in the pressure vessel. Accordingly, pressure may be applied to the fluid supplied to the membrane filtration apparatus in processing of the negatively charged membrane method, thereby reducing the time required for the processing of the negatively charged membrane method.

According to at least one embodiment, a concentration apparatus comprises: the membrane filtration apparatus described above; a sample water supply disposed upstream of the membrane filtration apparatus and configured to supply sample water; an acidic solution storage tank disposed upstream of the membrane filtration apparatus in parallel with the sample water supply and configured to store an acidic solution; an alkaline solution storage tank disposed upstream of the membrane filtration apparatus in parallel with the sample water supply and the acidic solution storage tank and configured to store an alkaline solution; an upstream pipe configured to supply the sample water from the sample water supply to the membrane filtration apparatus; a washing solution supply pipe branched from and connected to the upstream pipe and configured to supply a washing solution of strong acid to wash the membrane filtration apparatus; an outlet disposed downstream of the membrane filtration apparatus and configured to discharge fluid externally; and a collection container disposed downstream of the membrane filtration apparatus in parallel with the outlet and configured to collect the fluid. Accordingly, after treatment by the negatively charged membrane method using the concentration apparatus, the membrane filtration apparatus becomes usable again by washing with a strong acid. Therefore, convenience is increased by reducing the time and labor required to replace a filtration membrane, as well as reducing the cost of purchasing negatively charged membranes for replacement and the time taken from purchase to receipt.

According to an embodiment, the concentration apparatus further comprises a washing solution discharge pipe downstream of the membrane filtration apparatus and configured to discharge the washing solution. Accordingly, the washing solution of strong acid may be drained appropriately.

According to at least one embodiment, a concentration method is executed by a concentration apparatus comprising: a membrane filtration apparatus comprising a membrane that is made from an acid-resistant material, has pores having a pore size capable of capturing microorganisms, and has a surface that has a negative charge in a neutral to alkaline range; a sample water supply disposed upstream of the membrane filtration apparatus and configured to supply sample water; an acidic solution storage tank disposed upstream of the membrane filtration apparatus in parallel with the sample water supply and configured to store an acidic solution; an alkaline solution storage tank disposed upstream of the membrane filtration apparatus in parallel with the sample water supply and the acidic solution storage tank and configured to store an alkaline solution; an upstream pipe configured to supply the sample water from the sample water supply to the membrane filtration apparatus; a washing solution supply pipe branched from and connected to the upstream pipe and configured to supply a washing solution of strong acid to wash the membrane filtration apparatus; an outlet disposed downstream of the membrane filtration apparatus and configured to discharge fluid externally; and a collection container disposed downstream of the membrane filtration apparatus in parallel with the outlet and configured to collect the fluid, the concentration method comprising: filtering the sample water by supplying the sample water from the sample water supply to the membrane filtration apparatus; acid washing the membrane by supplying the acidic solution from the acidic solution storage tank to the membrane filtration apparatus; purifying a concentrate of microorganisms captured by the membrane by supplying the alkaline solution from the alkaline solution storage tank to the membrane filtration apparatus; and washing the membrane filtration apparatus by supplying the washing solution from the washing solution supply pipe to the membrane filtration apparatus. Accordingly, after treatment by the negatively charged membrane method using the concentration apparatus, the membrane filtration apparatus becomes usable again by washing with a strong acid. Therefore, convenience is increased by reducing the time and labor required to replace a filtration membrane, as well as reducing the cost of purchasing negatively charged membranes for replacement and the time taken from purchase to receipt.

### Advantageous Effect

According to the present disclosure, the membrane filtration apparatus, the concentration apparatus and the concentration method are provided that improve the convenience of processing by the negatively charged membrane method.

### BRIEF DESCRIPTION OF THE DRAWINGS

In the accompanying drawings:

FIG. 1 is a schematic diagram of a purification apparatus according to a comparative example, which purifies a concentrate of virus particles from sample water;

FIG. 2 is a schematic diagram for explanation of a processing step of a negatively charged membrane method executed via the equipment illustrated in FIG. 1;

FIG. 3 is a schematic diagram for explanation of a processing step of the negatively charged membrane method executed via the equipment illustrated in FIG. 1;

FIG. 4 is a schematic diagram for explanation of a processing step of the negatively charged membrane method executed via the equipment illustrated in FIG. 1;

FIG. 5 is a schematic diagram of a membrane filtration apparatus according to an embodiment of the present disclosure;

FIG. 6 is a schematic diagram of a concentration apparatus according to an embodiment of the present disclosure;

FIG. 7 is a schematic diagram for explanation of a first step of processing of the negatively charged membrane method executed by the concentration apparatus illustrated in FIG. 6;

FIG. 8 is a schematic diagram for explanation of a second step of processing of the negatively charged membrane method executed by the concentration apparatus illustrated in FIG. 6;

FIG. 9 is a schematic diagram for explanation of a third step of processing of the negatively charged membrane method executed by the concentration apparatus illustrated in FIG. 6; and

FIG. 10 is a schematic diagram for explanation of a fourth step of processing of the negatively charged membrane method executed by the concentration apparatus illustrated in FIG. 6.

### DETAILED DESCRIPTION

An embodiment of the present disclosure is described below with reference to the drawings.

First, a comparative example is described of equipment that purifies concentrates of microorganisms such as bacteria and viruses by a negatively charged membrane method. The negatively charged membrane method is an example of processing performed to purify a concentrate with increased virus concentration by concentrating viruses from sample water. Microorganisms such as bacteria and viruses are negatively charged in neutral to alkaline waters. Sample water subjected to the processing of the negatively charged membrane method is neutral to alkaline, and therefore such microorganisms are negatively charged in the sample water.

As an example, the present disclosure describes purification of a virus concentrate, but similar equipment and methods may be applied to other particulates such as bacteria. Viruses may be any known virus. Viruses may include, for example, double-stranded deoxyribonucleic acid (DNA) viruses, single-stranded DNA viruses, double-stranded ribonucleic acid (RNA) viruses, single-stranded RNA viruses, single-stranded RNA reverse transcription viruses, and double-stranded DNA reverse transcription viruses.

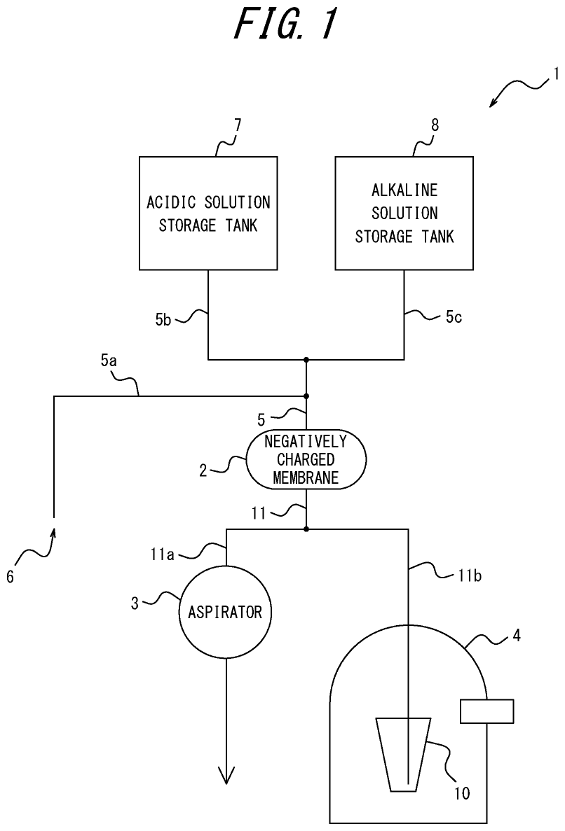

FIG. 1 is a schematic diagram for explanation of the comparative example of the equipment capable of performing processing by the negatively charged membrane method. As illustrated in FIG. 1, equipment 1 of the comparative example includes a negatively charged membrane 2, an aspirator 3, and a suction bottle 4. In FIG. 1, solid lines joining each component indicate piping through which fluid flows.

The negatively charged membrane 2 is a negatively charged membrane. For example, a mixed cellulose membrane manufactured by Millipore Corporation (hereinafter also referred to simply as “HA membrane”) may be used. The mixed cellulose membrane is, for example, a mixed membrane of nitrocellulose and cellulose acetate. The negatively charged membrane 2 has pores able to capture viruses and allow molecules that make up a fluid, such as water molecules, to permeate. The pore diameter of the pores in the negatively charged membrane 2 may be determined according to the viruses and the like to be captured by the negatively charged membrane 2.

A pipe 5 is provided upstream of the negatively charged membrane 2 to supply fluid. In the comparative example illustrated in FIG. 1, three different pipes 5a, 5b, and 5c are manually reconnected for each step, and different fluids are supplied to the negatively charged membrane 2 via the pipe 5. Instead of the pipes 5a, 5b, and 5c being reconnected to the pipe 5, different fluids may be supplied to the negatively charged membrane 2 via the pipe 5 by changing a solution fed into the pipe 5.

Of the three pipes 5a, 5b, and 5c, a first pipe 5a connects the pipe 5 to a sample water supply port 6. From the sample water supply port 6, sample water that may contain a virus is supplied to the first pipe 5a. The sample water supply port 6 may be supplied with a defined solution mixed with the sample water taken from a water treatment infrastructure facility. The defined solution may be, for example, a magnesium chloride solution. By supplying magnesium chloride solution, viruses in the sample water are agglomerated and more easily captured by the negatively charged membrane 2. As the defined solution, an appropriate solution may be used depending on properties of the sample water. Further, the defined solution need not be used, depending on the properties of the sample water.

Of the three pipes 5a, 5b, and 5c, a second pipe 5b connects the pipe 5 to an acidic solution storage tank 7 where an acidic aqueous solution is stored. The acidic aqueous solution is supplied to the second pipe 5b from the acidic solution storage tank 7. The acidic aqueous solution is described herein as a sulfuric acid solution as an example, but is not limited to this.

Of the three pipes 5a, 5b, and 5c, a third pipe 5c connects the pipe 5 to an alkaline solution storage tank 8 where an alkaline aqueous solution is stored. The alkaline aqueous solution is supplied to the third pipe 5c from the alkaline solution storage tank 8. The alkaline aqueous solution is described herein as a sodium hydroxide aqueous solution as an example, but is not limited to this.

Fluid is supplied to the negatively charged membrane 2 via the pipe 5 from each of the three pipes 5a, 5b, and 5c connected in a given step. Only one of the sample water, the acidic aqueous solution, and the alkaline aqueous solution is supplied to the negatively charged membrane 2 at a given time. In other words, the pipes 5a, 5b and 5c are manually reconnected for each step so that two or more of the sample water, the acidic aqueous solution, and the alkaline aqueous solution are not supplied at the same time.

The aspirator 3 is disposed downstream of the negatively charged membrane 2. The aspirator 3 draws in the fluid supplied to the negatively charged membrane 2 by creating a reduced pressure condition. In the comparative example illustrated in FIG. 1, in cases where the sample water and the acidic aqueous solution are supplied to the negatively charged membrane 2, the aspirator 3 is driven and fluid is drawn into the aspirator 3 and discharged externally.

The suction bottle 4 is disposed downstream of the negatively charged membrane 2 and in parallel with the aspirator 3. The suction bottle 4 draws in the fluid supplied to the negatively charged membrane 2 by creating a reduced pressure condition and collects the fluid in a concentrated fluid collection container 10 provided internally. In the comparative example illustrated in FIG. 1, when the alkaline aqueous solution is supplied to the negatively charged membrane 2, the fluid is drawn into the suction bottle 4 and collected in the concentrated fluid collection container 10.

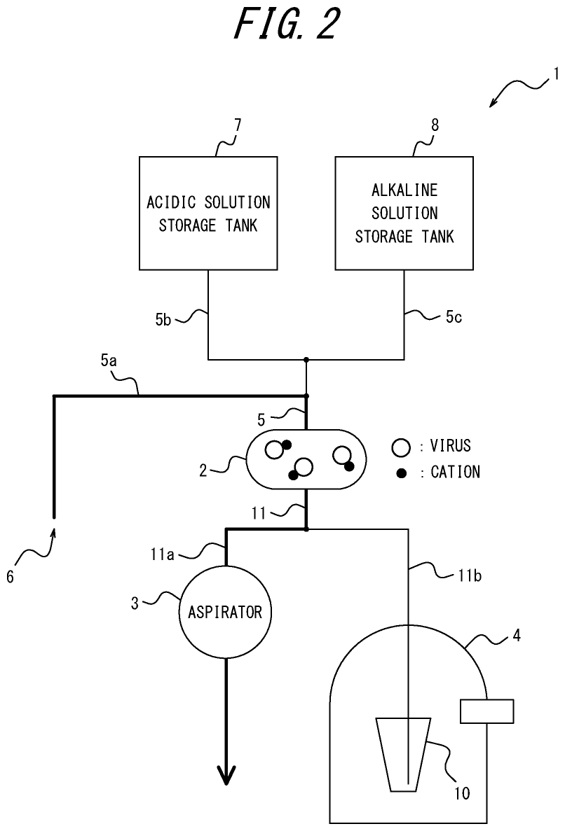

The following is a description of a processing method according to the negatively charged membrane method using the equipment of the comparative example illustrated in FIG. 1. FIG. 2 through FIG. 4 are schematic diagrams for explanation of processing steps of the negatively charged membrane method performed by the equipment 1 illustrated in FIG. 1. Bold lines in FIG. 2 through FIG. 4 indicate fluid flow. Here, a case is described where the sample water to be filtered is a small volume such as 2 liters or less. In this case, a flat membrane is used as the negatively charged membrane 2.

First, the first pipe 5a is connected to the pipe 5 and the aspirator 3 is driven to supply sample water from the sample water supply port 6 to the negatively charged membrane 2 via the first pipe 5a, as illustrated in FIG. 2. As the sample water passes through the negatively charged membrane 2, cations in the sample water have a positive charge and are therefore captured by the negatively charged membrane 2. Further, viruses in the sample water are larger than the pores of the negatively charged membrane 2 and are therefore captured by the negatively charged membrane 2. As the negatively charged membrane 2, one capable of capturing viruses at this time is used. For example, an HA membrane having a pore size of 0.45 μm and a diameter of 13 mm to 90 mm may be used as the negatively charged membrane 2. The sample water with captured cations and viruses is drained by the aspirator 3 through a pipe 11 and a pipe 11a disposed downstream of the negatively charged membrane 2.

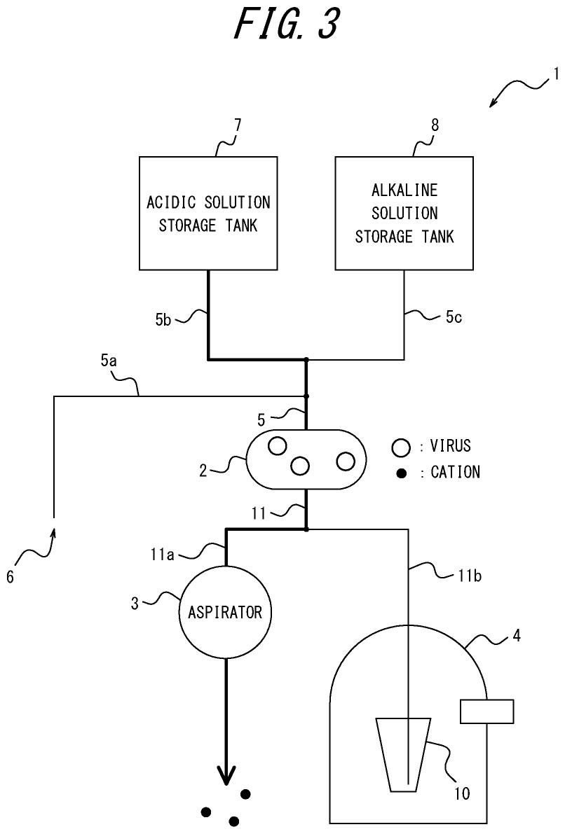

Next, the second pipe 5b is connected to the pipe 5 instead of the first pipe 5a, and the aspirator 3 is driven to supply the sulfuric acid solution from the acidic solution storage tank 7 through the second pipe 5b to the negatively charged membrane 2, as illustrated in FIG. 3. This results in acid washing of the negatively charged membrane 2. In other words, by supplying the sulfuric acid solution to the negatively charged membrane 2 and flowing downstream via the aspirator 3, the cations captured in the negatively charged membrane 2 are eluted from the negatively charged membrane 2 and drained with the sulfuric acid solution through the pipe 11 and the pipe 11a. The sulfuric acid solution may be any acid washing solution, for example 0.5 mM sulfuric acid solution at pH 3.0. The sulfuric acid solution may be supplied in an appropriate volume, for example, one-tenth or less of the volume of the supplied sample water. Acid washing leaves the negatively charged membrane 2 with viruses adhering thereto.

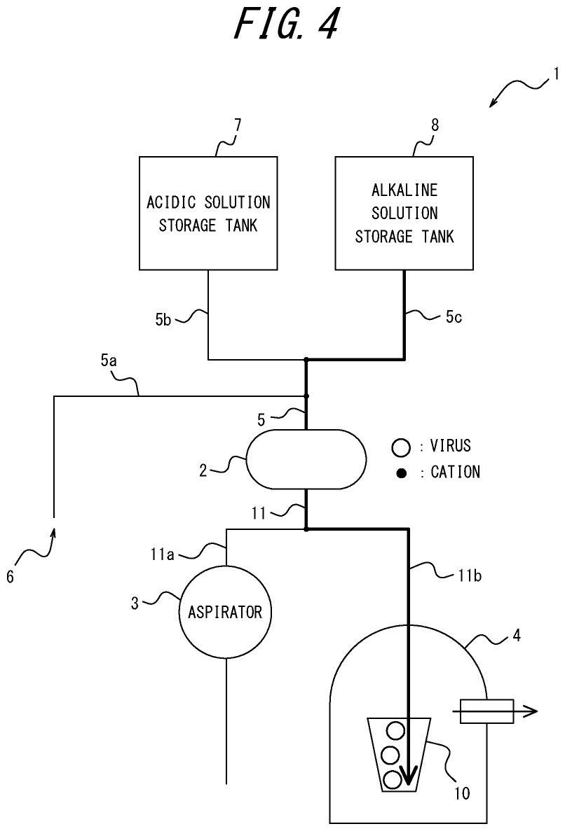

Then, the third pipe 5c is connected to the pipe 5 instead of the second pipe 5b, and sodium hydroxide aqueous solution is supplied to the negatively charged membrane 2 from the alkaline solution storage tank 8 through the third pipe 5c, as illustrated in FIG. 4. As a result, the negatively charged virus captured by the negatively charged membrane 2 is eluted from the negatively charged membrane 2 and flows with the sodium hydroxide aqueous solution through the pipe 11 and the pipe 11b to the suction bottle 4 and is collected in the concentrate collection container 10. The sodium hydroxide aqueous solution may be any solution from which viruses may be recovered, for example, a 1.0 mM sodium hydroxide aqueous solution at pH 10.5 to pH 10.8.

The sodium hydroxide aqueous solution may be supplied in an appropriate volume, for example, 1 ml to 10 ml. The smaller the amount of sodium hydroxide aqueous solution supplied, the higher the effect of virus concentration, which is preferable.

The concentrate collection container 10 is preferably pre-filled with a solution to neutralize the sodium hydroxide aqueous solution from which the virus is collected. For example, the concentrate collection container 10 is preferably pre-filled with 5 μl to 50 μl of 0.2 N sulfuric acid solution and 10 μl to 100 μl of pH 8.0 buffer solution.

Thus, the processing described with reference to FIG. 2 through FIG. 4 may purify a virus in the sample water into a concentrate collection container 10 by performing the negatively charged membrane method using the equipment 1.

When the sample water to be filtered is a large volume, for example, more than 2 liters, performing a two-step concentration using the negatively charged membrane 2 is preferable. When filtering a large volume of the sample water, a membrane having a housing shape is used as the negatively charged membrane 2. In the two-step concentration, an HA membrane having a pore size of 0.45 μm and a diameter of 293 mm, for example, or the Opticap® (Opticap is a registered trademark in Japan, other countries, or both) XL2 manufactured by Millipore Corporation, a cartridge-type filter, is used as the negatively charged membrane 2 in a first step primary concentration process, which is described in FIG. 2 through FIG. 4. Next, magnesium chloride solution is added to the primary concentrate purified in the primary concentration process in order to adsorb cations to the virus in the concentrate purified in the primary concentration process. The molar concentration and the volume of the magnesium chloride solution to be added may be determined based on the volume of the sample water, the primary concentrate, or the like. For example, 4.0 ml of 2.5 M magnesium chloride solution may be added. Next, a second step of secondary concentration is performed. In the secondary concentration process, the processing described in FIG. 2 through FIG. 4 is performed with respect to the primary concentrate to which the magnesium chloride solution is added. In this way, two-step concentration is performed.

In the negatively charged membrane method, a mixed cellulose membrane of nitrocellulose and cellulose acetate is generally used as the negatively charged membrane 2. The negatively charged membrane 2 is replaced each time filtration is performed to ensure filtration performance. However, the replacement of the negatively charged membrane 2 needs to be done manually, which requires time and labor. Further, the cost of purchasing the negatively charged membrane 2 for replacement and the time taken from purchase to receipt are required.

Further, when a flat membrane is used and raw sewage water with high turbidity and impurity concentration is used as the sample water, the filtration rate of filterable sample water is greatly reduced. In other words, when filtering the sample water with a high turbidity load, the filtration resistance in the negatively charged membrane 2 increases according to the amount of water permeating, and a situation may arise where sufficient filtration drive pressure by suction cannot be secured.

The following is a description of a membrane filtration apparatus, a concentration apparatus, and a concentration method that are able to solve the problems described above.

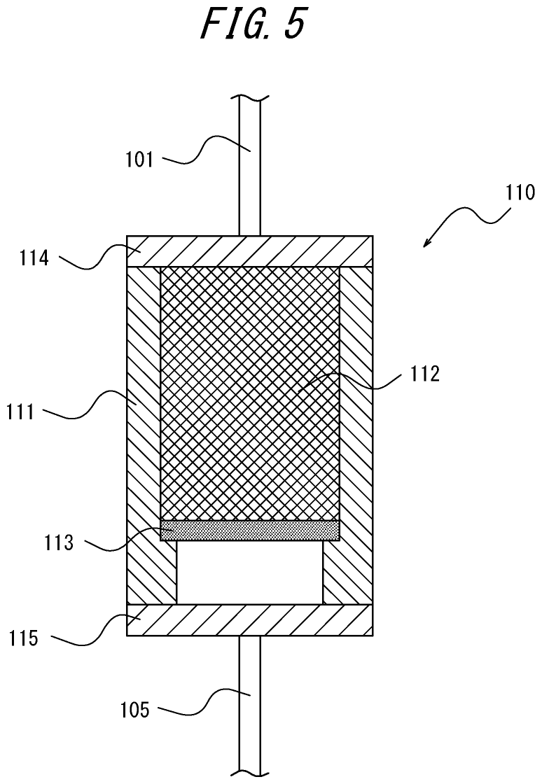

FIG. 5 is a schematic diagram of a membrane filtration apparatus 110 according to the embodiment of the present disclosure. The membrane filtration apparatus 110 illustrated in FIG. 5 is used in a concentration apparatus 100 described later. As illustrated in FIG. 5, the membrane filtration apparatus 110 includes a pressure vessel 111, a casing 112, a membrane 113, an upstream cover 114, and a downstream cover 115.

The pressure vessel 111 is a vessel that has a defined pressure resistance. The pressure vessel 111 has a pressure resistance of 0 MPa to 0.49 MPa, for example. However, the pressure resistance of the pressure vessel 111 is not limited to this example, and the pressure vessel 111 may be used that has pressure resistance appropriate for the specifications of the concentration apparatus 100 and other factors. The pressure vessel 111 may be configured to have a cylindrical shape, for example. The pressure vessel 111 holds the casing 112 inside.

The casing 112 is configured to have a cylindrical shape, for example, and is held by the pressure vessel 111. The casing 112 is a casing for securing the membrane 113 that is downstream, from upstream of the membrane 113. The casing 112 may be made from a material such as, but not limited to, glass, polypropylene, fluoroplastic, and the like, or any other suitable material.

The membrane 113 is held inside the pressure vessel 111 and secured downstream of the casing 112 by the casing 112. The membrane 113 is a membrane for virus capture. The membrane 113 is capable of membrane filtration at about pH 3 and a surface charge of the membrane 113 is preferably negative to near the isoelectric point. About pH 3 includes a range from pH 2.5 to pH 3.5, for example. The membrane 113 is capable of membrane filtration in a neutral to alkaline range. The neutral to alkaline range is, for example, pH 6 or higher. The membrane 113 has a negatively charged surface in the neutral to alkaline range. Accordingly, the membrane 113 functions as a negatively charged membrane in the neutral to alkaline range.

The membrane 113 is composed of acid-resistant material. The membrane 113 is particularly preferably composed of acid-resistant material able to withstand pH 2 or lower. Accordingly, the membrane filtration apparatus 110 is less susceptible to erosion by a solution of strong acid when the membrane filtration apparatus 110 is washed with the solution of strong acid, as described below, and the charge is not degraded even when the device is washed with the washing solution as described below.

The membrane 113 may be made from an organic membrane material such as polyethylene, tetrafluoroethylene, polypropylene, cellulose acetate, polyacrylonitrile, polyimide, polysulfone, polyethersulfone, and the like. The membrane 113 may be made from a ceramic membrane using aluminum oxide, zirconium oxide, titanium oxide, and the like, or an inorganic membrane material such as stainless steel, glass, and the like.

The membrane 113 has pores having an appropriate pore size capable of capturing viruses. The membrane 113 may have pores 0.1 μm to 2 μm in diameter, for example. The membrane 113 may be a microfiltration membrane. The membrane 113 may have a flat membrane shape or a housing shape. The membrane 113 may be configured as a cartridge-type filter.

The pressure vessel 111 and the casing 112 have appropriate dimensions and shape according to the shape of the membrane 113, bore diameter, and the like. Further, the membrane filtration apparatus 110 may be provided with a jig downstream of the membrane 113 to fix the membrane 113 to the pressure vessel 111. In this case, the jig has a mesh or porous structure.

The upstream cover 114 and the downstream cover 115 are covers covering the upstream and downstream sides of the pressure vessel 111, respectively. The upstream cover 114 and the downstream cover 115 are pressure-resistant. The upstream cover 114 and the downstream cover 115 may be fixed to the pressure vessel 111 by any method, such as by being screwed or jigged, for example.

As illustrated in FIG. 5, the upstream pipe 101 is connected upstream of the membrane filtration apparatus 110. Further, the downstream pipe 105 is connected downstream of the membrane filtration apparatus 110. When the sample water is supplied from the upstream pipe 101 to the membrane filtration apparatus 110, the sample water is filtered by viruses being captured by the membrane 113 of the membrane filtration apparatus 110, and the filtered water is discharged from the downstream pipe 105. Discharge is performed, for example, by suctioning with a suction pump provided downstream of the downstream pipe 105.

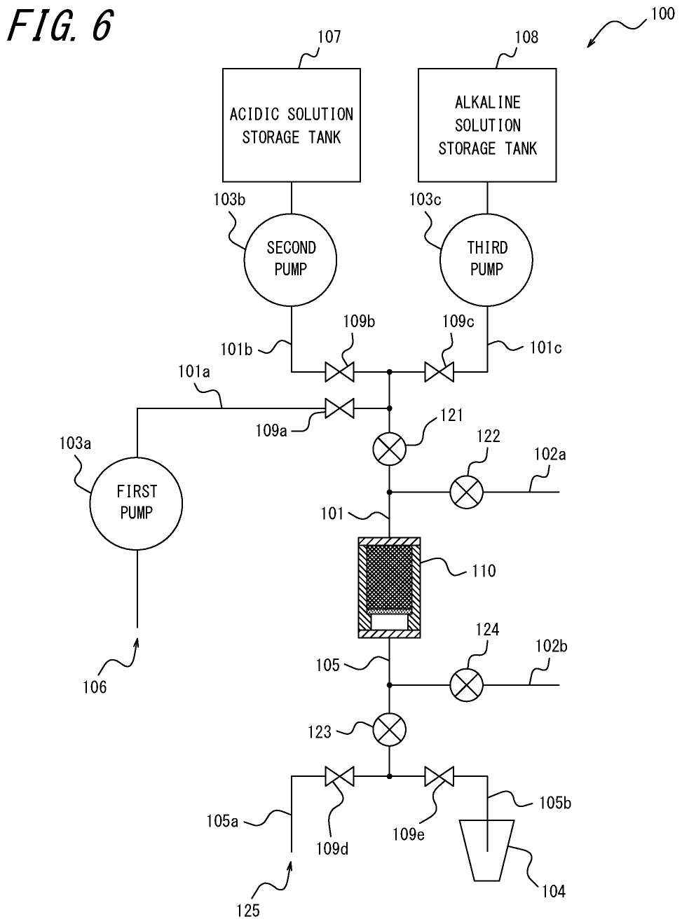

FIG. 6 is a schematic diagram of the concentration apparatus 100 according to an embodiment of the present disclosure. As illustrated in FIG. 6, the concentration apparatus 100 includes the membrane filtration apparatus 110, a first pump 103a, a second pump 103b, a third pump 103c, and a concentrate collection container 104. In FIG. 6, solid lines joining each component indicate piping through which fluid flows.

Upstream of the membrane filtration apparatus 110 is an upstream pipe 101 for supplying fluid. A washing solution supply pipe 102a is branched from and connected to the upstream pipe 101 to supply solution to wash the membrane filtration apparatus 110. The upstream pipe 101 is provided with a first gate valve 121 that controls a fluid supply route upstream of the junction with the washing solution supply pipe 102a. The washing solution supply pipe 102a is provided with a second gate valve 122 that controls a supply route of the washing solution to the membrane filtration apparatus 110. Upstream of the washing solution supply pipe 102a is a washing solution storage tank (not illustrated) that stores the washing solution.

Three different pipes, a first pipe 101a, a second pipe 101b, and a third pipe 101c, are connected to the upstream pipe 101, upstream of the first gate valve 121, and different fluids are supplied from these three pipes 101a, 101b, and 101c, respectively, to the membrane filtration apparatus 110 via the upstream pipe 101.

The first pipe 101a connects the upstream pipe 101 to the sample water supply 106. From the sample water supply 106, the sample water that may contain a virus is supplied to the first pipe 101a. The sample water supply 106 may be supplied with a defined solution mixed with the sample water taken from a water treatment infrastructure facility, similar to that described for the comparative example. The defined solution is a magnesium chloride solution. By mixing the magnesium chloride solution, viruses in the sample water are agglomerated and more easily captured by the membrane 113. A valve 109a is provided to the first pipe 101a. Opening and closing of the valve 109a controls the supply of the sample water from the first pipe 101a.

The first pipe 101a is provided with a first pump 103a. The first pump 103a pumps the sample water downstream from the sample water supply 106. The first pump 103a may be a pressure pump. For example, use of a pressure pump as the first pump 103a is preferred when filtering a small volume of the sample water that is a high load for the membrane filtration apparatus 110 due to high turbidity and insoluble substance concentration in the sample water. By using a pressure pump, the sample water is supplied at higher pressure and preventing precipitation of turbid or insoluble substances in the pipe is made easier. Further, use of a pressure pump as the first pump 103a is preferred when filtering a large volume of the sample water. By using a pressure pump, the sample water is supplied at higher pressure and the time required for filtration is reduced.

The second pipe 101b connects the upstream pipe 101 to the acidic solution storage tank 107. The acidic solution storage tank 107 is a tank that stores an acidic aqueous solution (acidic solution). The acidic aqueous solution is described herein as a sulfuric acid solution as an example, but is not limited to this. A valve 109b is provided to the second pipe 101b. Opening and closing of the valve 109b controls the supply of the acidic solution from the second pipe 101b.

The second pipe 101b is provided with a second pump 103b. The second pump 103b pumps the acidic solution downstream from the acidic solution storage tank 107. The second pump 103b may be a pressure pump.

The third pipe 101c connects the upstream pipe 101 to the alkaline solution storage tank 108. The alkaline solution storage tank 108 is a tank that stores an alkaline aqueous solution (alkaline solution). The alkaline aqueous solution is described herein as a sodium hydroxide aqueous solution as an example, but is not limited to this. A valve 109c is provided to the third pipe 101c. Opening and closing of the valve 109c controls the supply of the alkaline solution from the third pipe 101c.

The third pipe 101c is provided with a third pump 103c. The third pump 103c pumps the alkaline solution downstream from the alkaline solution storage tank 108. The third pump 103c may be a pressure pump.

The membrane filtration apparatus 110 is the membrane filtration apparatus 110 described with reference to FIG. 5. The membrane filtration apparatus 110 may be provided with a flat membrane having a diameter of about 10 mm, for example, as the membrane 113.

Downstream of the membrane filtration apparatus 110 is the downstream pipe 105 for discharging fluid that has passed through the membrane filtration apparatus 110. A washing solution discharge pipe 102b is branched from and connected to the downstream pipe 105 to discharge the washing solution used for washing the membrane filtration apparatus 110. The downstream pipe 105 is provided with a third gate valve 123 that controls a fluid discharge route downstream from the junction with the washing solution discharge pipe 102b. The washing solution discharge pipe 102b is provided with a fourth gate valve 124 that controls a discharge route of the washing solution. A washing solution collection tank (not illustrated) may be provided downstream of the washing solution discharge pipe 102b to collect the washing solution discharged from the membrane filtration apparatus 110.

Two different pipes, a fourth pipe 105a and a fifth pipe 105b, are connected to the downstream pipe 105 downstream of the third gate valve 123.

The fourth pipe 105a is connected to an outlet 125 and discharges fluid from the outlet 125. The fourth pipe 105a is provided with a valve 109d. Opening and closing of the valve 109d controls the discharge of fluid from the fourth pipe 105a.

The fifth pipe 105b connects the downstream pipe 105 to the concentrate collection container 104. The concentrate collection container 104 is a container for collecting the virus concentrate. The fifth pipe 105b is provided with a valve 109e. Opening and closing of the valve 109e controls the pumping of concentrate from the fifth pipe 105b to the concentrate collection container 104.

Each component of the concentration apparatus 100 has a defined pressure resistance, such as 0.49 MPa, for example. Further, the drive pressure in the concentration apparatus 100 is determined based on the pressure resistance of the membrane 113 of the membrane filtration apparatus 110, which may be up to 0.3 MPa, for example. The drive pressure is the difference between the feed pressure of the sample water to the membrane filtration apparatus 110 and the permeate pressure of the sample water that permeates through the membrane filtration apparatus 110.

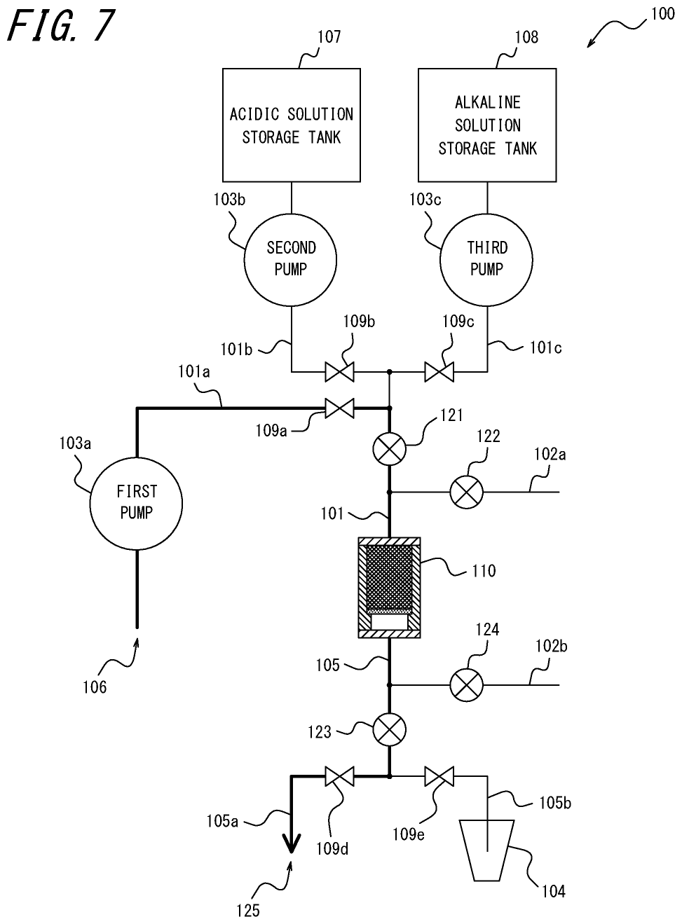

Next, processing steps of the negatively charged membrane method using the concentration apparatus 100 illustrated in FIG. 6 are described with reference to FIG. 7 through FIG. 10. FIG. 7 through FIG. 10 are schematic diagrams for explanation of steps of processing the negatively charged membrane method executed by the concentration apparatus 100. Bold lines in FIG. 7 through FIG. 10 indicate fluid flow. In the concentration apparatus 100, purification of the concentrate of virus particles by the negatively charged membrane method is performed in first through third steps, and washing of the membrane 113 provided to the membrane filtration apparatus 110 is performed in a fourth step.

FIG. 7 is a schematic illustration for explanation of the first step of the processing of the negatively charged membrane method executed by the concentration apparatus 100 illustrated in FIG. 6. The first step is to filter the sample water.

In the first step, the valve 109a, the valve 109d, the first gate valve 121, and the third gate valve 123 are open, and the valve 109b, the valve 109c, the valve 109e, the second gate valve 122, and the fourth gate valve 124 are closed. As a result, the sample water that may contain a virus is supplied from the sample water supply 106 to the membrane filtration apparatus 110 via the first pipe 101a and the upstream pipe 101. The sample water supplied to the membrane filtration apparatus 110 is filtered through the membrane 113 of the membrane filtration apparatus 110. In other words, the cations in the sample water have a positive charge and are therefore captured by the negatively charged membrane 113. Further, viruses in the sample water are larger than the pores of the membrane 113 and are therefore captured by the membrane 113. The sample water after being filtered by the membrane 113 is drained through the downstream pipe 105 and the fourth pipe 105a.

Filtration in the first step may be performed over a period of time, such as from a few seconds to an hour. In the first step, the sample water may be pumped downstream by the first pump 103a. The drive pressure of the first pump 103a may be adjusted appropriately within the allowable pressure of the concentration apparatus 100. The higher the drive pressure of the first pump 103a, the shorter the time required for the filtration of the first step.

The sample water may be pumped downstream by a pressure-reducing pump instead of by pressure from the first pump 103a. In this case, the pressure-reducing pump is disposed downstream of the membrane filtration apparatus 110. The sample water may be pumped downstream by application of pressure using a pressure tank. In a case where a pressure-reducing pump or pressure tank is used, the time required for the first step of filtration is also reduced. By any of these methods, the filtration resistance at the membrane 113 increases according to Darcy's law, depending on the time required for filtration and the volume of the sample water to be filtered, and therefore when filtration continues at the same pressure, the volume of water filtered may gradually decrease. With this in mind, the drive pressure of the pump or pressure tank may be increased according to the time required or the volume of the sample water filtered, for example. Further, when the sample water has a high turbidity or concentration of an insoluble substance, or contains a high amount of dissolved organic matter, such as sewage or treated sewage for example, the filtration resistance will gradually increase. In this case, the drive pressure of the pump or pressure tank may be increased gradually.

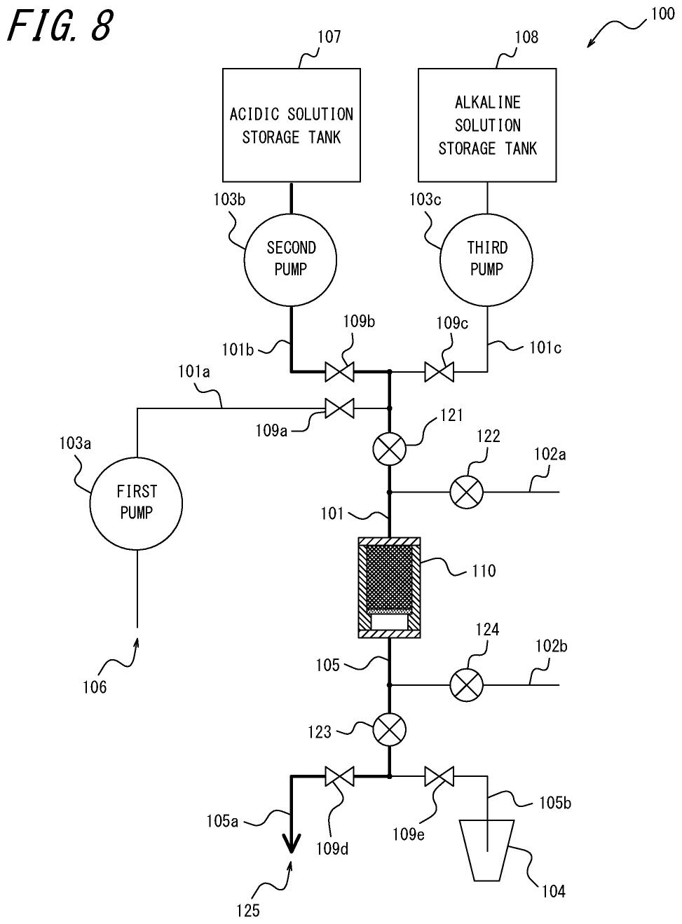

FIG. 8 is a schematic illustration for explanation of the second step of the processing of the negatively charged membrane method executed by the concentration apparatus 100 illustrated in FIG. 6. The second step is to acid wash the membrane 113.

In the second step, the valve 109b, the valve 109d, the first gate valve 121, and the third gate valve 123 are open, and the valve 109a, the valve 109c, the valve 109e, the second gate valve 122, and the fourth gate valve 124 are closed. In this state, the second pump 103b is driven to supply sulfuric acid solution from the acidic solution storage tank 107 to the membrane filtration apparatus 110 via the second pipe 101b and the upstream pipe 101 to perform acid washing of the membrane 113. In other words, the cations captured by the membrane 113 in the first step are eluted from the membrane 113 and drained along with the sulfuric acid solution through the downstream pipe 105 and the fourth pipe 105a. In this way, the cations are removed from the membrane 113.

The sulfuric acid solution may be, for example, a 0.5 mM sulfuric acid solution at pH 3.0. The amount of the sulfuric acid solution supplied is preferably just enough that the sulfuric acid solution evenly contacts the entire surface of the membrane 113. For example, when filtering a small volume of the sample water, the amount of the sulfuric acid solution supplied may be one-tenth the volume of the sample water or 10 mL or more. When filtering a large volume of the sample water, the amount of the sulfuric acid solution supplied may be one-tenth the volume of the sample water.

The concentration apparatus 100 may be configured to include a valve downstream of the membrane 113 to control the time or state of contact and immersion of the sulfuric acid solution with the membrane. In order that the membrane 113 is evenly immersed in the sulfuric acid solution, when the membrane 113 has a flat membrane structure, the sulfuric acid solution feed is preferably controlled so that the sulfuric acid solution is uniformly spread to a depth of a few millimeters over the membrane surface. When the membrane 113 has a housing shape, the sulfuric acid solution feed is preferably controlled so that the sulfuric acid solution is spread on the upstream side of the membrane filtration apparatus 110. The membrane 113 is preferably immersed in sulfuric acid solution for a few seconds or more. Accordingly, the amount of cations remaining in the membrane 113 is reduced, and more appropriate acid washing is performed.

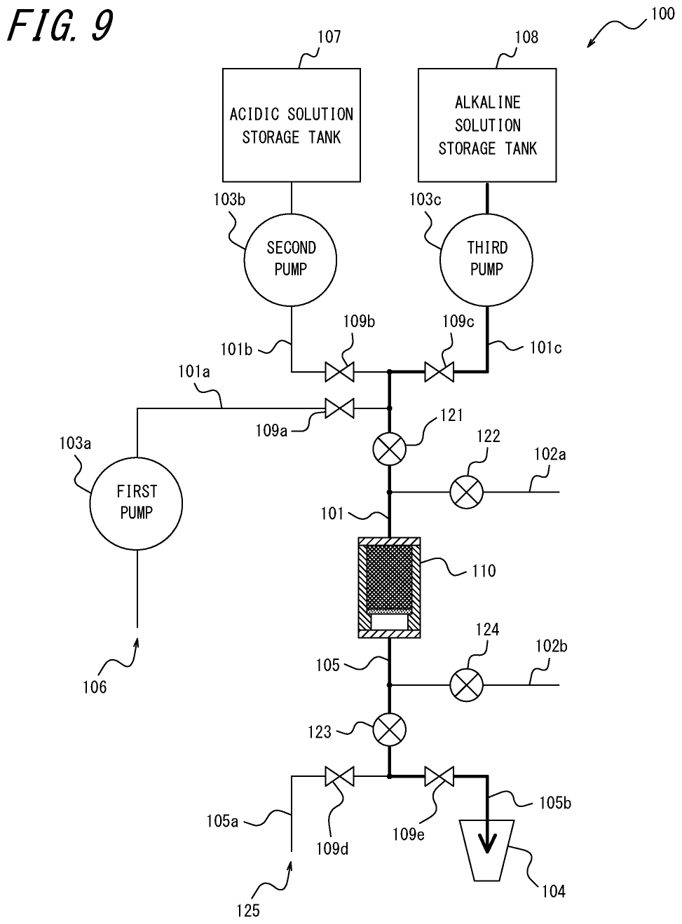

FIG. 9 is a schematic illustration for explanation of the third step of the processing of the negatively charged membrane method executed by the concentration apparatus 100 illustrated in FIG. 6. The third step is to purify the virus concentrate by alkaline elution.

In the third step, the valve 109c, the valve 109e, the first gate valve 121, and the third gate valve 123 are open, and the valve 109a, the valve 109b, the valve 109d, the second gate valve 122, and the fourth gate valve 124 are closed. In this state, the third pump 103c is driven to supply sodium hydroxide aqueous solution from the alkaline solution storage tank 108 to the membrane filtration apparatus 110 via the third pipe 101c and the upstream pipe 101 to perform alkaline elution of the membrane 113. In other words, the virus captured by the membrane 113 in the first step is eluted from the membrane 113 and flows downstream through the downstream pipe 105 and the fifth pipe 105b together with the sodium hydroxide aqueous solution, and the concentrate of the virus is collected in the concentrate collection container 104.

For example, a 1.0 mM sodium hydroxide aqueous solution at pH 10.5 to pH 10.8 may be used. The sodium hydroxide aqueous solution supplied may be, for example, 10 ml or more. However, the smaller the amount of sodium hydroxide aqueous solution supplied, the higher the effect of virus concentration, which is preferable.

The concentration apparatus 100 may be configured to include a valve downstream of the membrane 113 to control the time or state of contact and immersion of the sodium hydroxide aqueous solution with the membrane. In order that the membrane 113 is evenly immersed in the sodium hydroxide aqueous solution, when the membrane 113 has a flat membrane structure, the sodium hydroxide aqueous solution feed is preferably controlled so that the sodium hydroxide aqueous solution is uniformly spread to a depth of a few millimeters over the membrane surface. When the membrane 113 has a housing shape, the sodium hydroxide aqueous solution feed is preferably controlled so that the sodium hydroxide aqueous solution is spread on the upstream side of the membrane filtration apparatus 110. The membrane 113 is preferably immersed in sodium hydroxide aqueous solution for a few seconds or more. This allows more virus to be eluted from the membrane 113 and collected as a virus concentrate.

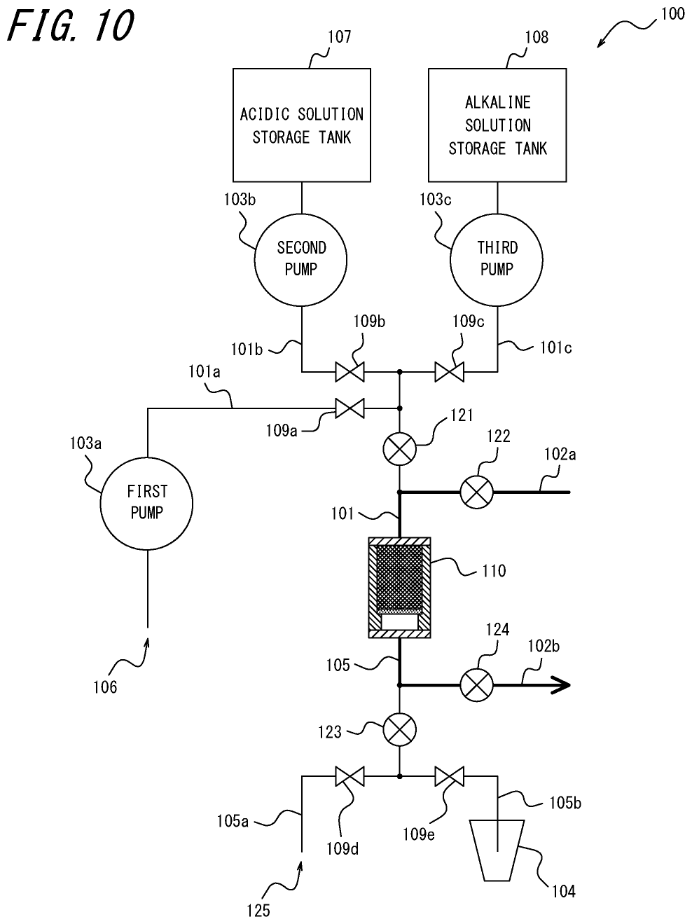

FIG. 10 is a schematic illustration for explanation of the fourth step of the processing of the negatively charged membrane method executed by the concentration apparatus 100 illustrated in FIG. 6. The fourth step is to wash the membrane filtration apparatus 110.

In the fourth step, the second gate valve 122 and the fourth gate valve 124 are open, and the first gate valve 121 and the third gate valve 123 are closed. The washing solution is supplied from the washing solution storage tank to the membrane filtration apparatus 110 via the washing solution supply pipe 102a. After passing through the membrane filtration apparatus 110, the washing solution is discharged from the washing solution discharge pipe 102b. In this way, the washing solution is appropriately drained.

The washing solution is a solution of strong acid. The washing solution is preferably a solution of strong acid having a pH of 2 or lower. By supplying the washing solution to the membrane filtration apparatus 110, the ability to filter by the membrane filtration apparatus 110 may be restored after the processing by the negatively charged membrane method in the first through third steps. Specifically, nucleic acids such as RNA and DNA of a virus remaining in the membrane filtration apparatus 110 may be dissolved by the supply of the washing solution of strong acid to the membrane filtration apparatus 110. Accordingly, the membrane filtration apparatus 110 is washed, and therefore the same membrane filtration apparatus 110 may be used for subsequent processing without contamination or other effects on a sample used in the subsequent processing. In other words, the membrane filtration apparatus 110 is ready for use again. In particular, the membrane 113 of the membrane filtration apparatus 110 is made of acid-resistant material, and therefore the washing solution of strong acid is able to remove nucleic acids derived from viruses without damaging the membrane 113.

The washing solution may be supplied from an appropriate location of the concentration apparatus 100. The washing solution is preferably supplied from at least upstream of the membrane filtration apparatus 110. Accordingly, the membrane filtration apparatus 110 may be washed.

Thus, the concentration apparatus 100 according to the present embodiment uses the membrane filtration apparatus 110 that includes the membrane 113 that is made from an acid-resistant material and has a surface that has a negative charge in a neutral to alkaline range. The membrane 113 functions as a negatively charged membrane during processing according to the negatively charged membrane method. After the processing according to the negatively charged membrane method is completed, virus-derived substances (for example, nucleic acids) may be dissolved by supplying the washing solution of strong acid to the membrane filtration apparatus 110. Accordingly, the membrane filtration apparatus 110 is washed, and therefore the same membrane filtration apparatus 110 may be used for subsequent processing without contamination or other effects on a sample used in the subsequent processing. In this way, after the processing according to the negatively charged membrane method, the concentration apparatus 100 is washed and ready for use again. In other words, according to the concentration apparatus 100, the membrane 113 for filtration may be washed and reused while still attached. Accordingly, the membrane filtration apparatus 110 may be used repeatedly. Therefore, convenience is increased by reducing the time and labor required to replace a filtration membrane, as well as reducing the cost of purchasing negatively charged membranes for replacement and the time taken from purchase to receipt.

The processing of the first through fourth steps by the concentration apparatus 100 according to the above embodiment may be automated. For example, the concentration apparatus 100 may include a controller capable of controlling the first pump 103a, the second pump 103b, the third pump 103c, the valves 109a through 109e, the first gate valve 121 through to the fourth gate valve 124, and the like, and a series of processes from the first step to the fourth step may be realized by control of these mechanisms by the controller. Such automation eliminates the need to arrange for specialized workers to purify the virus concentrate using the concentration apparatus 100. Further, because manual labor may be eliminated, differences due to individual differences in the processing of the negatively charged membrane method may be eliminated, and the quality of the processing may be made uniform.

Further, according to the embodiment described above, the membrane filtration apparatus 110 is described as being washed by supply of the washing solution to the membrane filtration apparatus 110. However, washing with the washing solution of strong acid may be performed on other mechanisms constituting the concentration apparatus 100. Washing with the washing solution of strong acid may be performed on any mechanism affected by the sample water. For example, the washing solution of strong acid may be supplied to the upstream pipe 101 and the first pipe 101a, or to the first pump 103a, which supply the sample water, to wash these mechanisms.

The above embodiment describes an example in which the concentration apparatus 100 automatically controls purification of a concentrate of microorganisms such as bacteria and viruses, but the concentration apparatus 100 is equally applicable to particulate and colloidal dispersions that are negatively charged and in solution.

The concentration apparatus 100 described above may be used in a variety of fields and applications. For example, the concentration apparatus 100 described above may be used to monitor water quality management and treatment performance of water treatment infrastructure such as water purification plants, sewage treatment plants, water reclamation facilities, and seawater desalination facilities. Further, the concentration apparatus 100 described above may be used to determine the treatment performance of water treatment systems that make up water treatment infrastructure, such as coagulation tanks, sedimentation tanks, sand filtration, microfiltration membranes, ultrafiltration membranes, reverse osmosis membranes, ozone contact tanks, activated carbon filtration tanks, ultraviolet (UV) irradiation tanks, disinfection tanks using chlorine agents, and the like. The concentration apparatus 100 described above may also be used, for example, to conduct dynamic surveys of the environment in water bodies such as rivers, oceans, water features, swimming pools, bathing areas, and the like. The concentration apparatus 100 described above may also be used, for example, for water quality testing for particulate and colloidal dispersion, microorganisms, and the like to determine the risk of microbial infection in a city including water bodies and environmental infrastructure. The concentration apparatus 100 described above may also be used for the purpose of qualitative risk, safety monitoring, or quality control of liquids used for beverages or in the production of processed foods, to quantify risk or to verify a comparison with a threshold value to determine safety. The concentration apparatus 100 described above may also be used for testing the quality of water for industrial, irrigation, and agricultural use. The concentration apparatus 100 described above may be used, for example, to manage qualitative risk, safety monitoring, or quality control of liquids used for temperature and humidity management, such as in mist spraying, humidification devices, or water sprinkling. The concentration apparatus 100 described above may also be used with respect to water for which the use of permanent water quality research facilities is restricted, such as water for emergency or disaster situations. The concentration apparatus 100 described above may also be used to test the quality of water used in vehicles or transportation systems with attached living facilities, such as campers, large buses, ships, submarines, aircraft, space stations, and the like. The concentration apparatus 100 described above may also be used for quality control testing of water related to medical care such as water used in pharmaceutical manufacturing, dialysis therapy, and the like.

The present disclosure is not limited to the configurations specified in the embodiments described above, and various modifications are possible without departing from the scope of the claims. For example, functions included in each component, each step, and the like may be reconfigured and multiple components or steps may be combined into one or divided, as long as no logical inconsistency results.

### REFERENCE SIGNS LIST

1 Equipment
2 Negatively charged membrane
3 Aspirator
4 Suction bottle
5, 11, 11a, 11b Pipe
5a, 101a First pipe
5b, 101b Second pipe
5c, 101c Third pipe
6 Sample water supply port
7, 107 Acidic solution storage tank
8, 108 Alkaline solution storage tank
9a, 9b, 9c, 109a, 109b, 109c, 109d, 109e Valve
10, 104 Concentrate collection container
100 Concentration apparatus
101 Upstream pipe
102a Washing solution supply pipe
102b Washing solution discharge pipe
103a First pump
103b Second pump
103c Third pump
105 Downstream pipe
105a Fourth pipe
105b Fifth pipe
106 Sample water supply
110 Membrane filtration apparatus
111 Pressure vessel
112 Casing
113 Membrane
114 Upstream cover
115 Downstream cover
121 First gate valve
122 Second gate valve
123 Third gate valve
124 Fourth gate valve
125 Outlet

1. A membrane filtration apparatus comprises a membrane that is made from an acid-resistant material, has pores having a pore size capable of capturing microorganisms, and has a surface that has a negative charge in a neutral to alkaline range.

2. The membrane filtration apparatus according to claim 1, wherein the membrane is made from an acid-resistant material that withstands pH 2 or lower.

3. The membrane filtration apparatus according to claim 1, further comprising:
a pressure vessel that is pressure resistant, wherein
the membrane is held in the pressure vessel.

4. A concentration apparatus comprising:
the membrane filtration apparatus according to claim 1;
a sample water supply disposed upstream of the membrane filtration apparatus and configured to supply sample water;
an acidic solution storage tank disposed upstream of the membrane filtration apparatus in parallel with the sample water supply and configured to store an acidic solution;
an alkaline solution storage tank disposed upstream of the membrane filtration apparatus in parallel with the sample water supply and the acidic solution storage tank and configured to store an alkaline solution;
an upstream pipe configured to supply the sample water from the sample water supply to the membrane filtration apparatus;
a washing solution supply pipe branched from and connected to the upstream pipe and configured to supply a washing solution of strong acid to wash the membrane filtration apparatus;
an outlet disposed downstream of the membrane filtration apparatus and configured to discharge fluid externally; and
a collection container disposed downstream of the membrane filtration apparatus in parallel with the outlet and configured to collect fluid.

5. The concentration apparatus according to claim 4, further comprising a washing solution discharge pipe downstream of the membrane filtration apparatus and configured to discharge the washing solution.

6. A concentration method executed by
a concentration apparatus comprising:
a membrane filtration apparatus comprising a membrane that is made from an acid-resistant material, has pores having a pore size capable of capturing microorganisms, and has a surface that has a negative charge in a neutral to alkaline range;
a sample water supply disposed upstream of the membrane filtration apparatus and configured to supply sample water;
an acidic solution storage tank disposed upstream of the membrane filtration apparatus in parallel with the sample water supply and configured to store an acidic solution;
an alkaline solution storage tank disposed upstream of the membrane filtration apparatus in parallel with the sample water supply and the acidic solution storage tank and configured to store an alkaline solution;
an upstream pipe configured to supply the sample water from the sample water supply to the membrane filtration apparatus;
a washing solution supply pipe branched from and connected to the upstream pipe and configured to supply a washing solution of strong acid to wash the membrane filtration apparatus;
an outlet disposed downstream of the membrane filtration apparatus and configured to discharge fluid externally; and
a collection container disposed downstream of the membrane filtration apparatus in parallel with the outlet and configured to collect fluid,
the concentration method comprising:
filtering the sample water by supplying the sample water from the sample water supply to the membrane filtration apparatus;
acid washing the membrane by supplying the acidic solution from the acidic solution storage tank to the membrane filtration apparatus;
purifying a concentrate of microorganisms captured by the membrane by supplying the alkaline solution from the alkaline solution storage tank to the membrane filtration apparatus; and
washing the membrane filtration apparatus by supplying the washing solution from the washing solution supply pipe to the membrane filtration apparatus.
